(19) United States
(12) Patent Application Publication
(10) Pub. No.: US 2024/0047047 A1
(43) Pub. Date: Feb. 8, 2024

(54) BIOLOGICAL INFORMATION MANAGEMENT SYSTEM, RELAY DEVICE, AND BIOLOGICAL INFORMATION MANAGEMENT METHOD

(71) Applicant: Murata Manufacturing Co., Ltd., Nagaokakyo-shi (JP)

(72) Inventor: Hirofumi TSUCHIMOTO, Nagaokakyo-shi (JP)

(21) Appl. No.: 18/489,162

(22) Filed: Oct. 18, 2023

Related U.S. Application Data
(63) Continuation of application No. PCT/JP2022/013020, filed on Mar. 22, 2022.

(30) Foreign Application Priority Data
Jun. 8, 2021 (JP) 2021-095850

(51) Int. Cl. G16H 40/00; G16H 10/65; A61B 5/00; A61B 5/0245

(52) U.S. Cl. CPC G16H 40/00; G16H 10/65; A61B 5/742; A61B 5/0245

(57) ABSTRACT

A biological information management system is provided with enhanced security. The biological information management system includes a biological information sensor that measures time-series waveform data of an electric signal related to biological information of a user and transmits the measured time-series waveform data of the electric signal and a sensor ID. The system further includes a relay device that transmits the time-series waveform data of the electric signal and a sensor-related ID related to the sensor ID without attaching a user ID, which is personal information, to the time-series waveform data of the electric signal and the sensor-related ID. A server device stores the time-series waveform data of the electric signal and the sensor-related ID received from the relay device without attaching the user ID, which is the personal information, to the time-series waveform data of the electric signal and the sensor-related ID.

### CROSS REFERENCE TO RELATED APPLICATIONS

This application is a continuation of International Application No. PCT/JP2022/013020, filed Mar. 22, 2022, which claims priority to Japanese Patent Application No. 2021-095850, filed Jun. 8, 2021, the entire contents of each of which are hereby incorporated by reference in their entirety.

### TECHNICAL FIELD

The present invention relates to a biological information management system, a relay device, and a biological information management method.

### BACKGROUND

Japanese Patent No. 6620456 (hereinafter “Patent Document 1”) describes an information processing system (e.g., a biological information management system) including a wearable device (e.g., a biological information sensor) that measures medical physical information including biological information, such as vital information or vital signs, of a user, a gateway device (e.g., a relay device), such as a smartphone, and a server that stores physical information of the user received from the wearable device via the gateway device. Also, the wearable device stores, in advance, physical characteristics information of the user including personal information of the user such as a name, an address, and a birth date and information related to health and medical care of the user (e.g., information related to illnesses, diseases, and treatments). The server also stores the physical characteristics information of the user received from the wearable device. This configuration provides optimum information for each user according to the physical information and the physical characteristics information of the user in the event of a disaster.

The personal information of the user, such as the name, the address, and the birth date of the user, corresponds to personal information defined in the Act on the Protection of Personal Information. The medical physical information including biological information (e.g., vital information or vital signs) of the user and the information related to health and medical care of the user (e.g., information related to illnesses, diseases, and treatments) correspond to medical information that may correspond to sensitive personal information defined in the Act on the Protection of Personal Information.

However, the information processing system described in Patent Document 1 has a problem in terms of information protection in times other than an emergency such as a disaster. For example, there are risks such as an information leak, unauthorized viewing (peeping), and tampering of data relayed by the gateway device such as a smartphone. Similarly, for example, there are risks such as an information leak, unauthorized viewing, and tampering of data stored in the server.

### SUMMARY OF THE INVENTION

Accordingly, the exemplary aspects of the present invention provide a biological information management system, a relay device, and a biological information management method with enhanced security.

According to an exemplary aspect, a biological information management system is provided that manages a measurement result related to biological information of a user and obtained by a biological information sensor. The biological information management system includes the biological information sensor that measures time-series waveform data of an electric signal related to the biological information of the user and transmits the measured time-series waveform data of the electric signal and a sensor ID for identifying the biological information sensor. In this aspect, the time-series waveform data of the electric signal is medical information unprocessed data that has not been processed into the biological information that is medical information as well as sensitive personal information. The system includes a relay device that receives the time-series waveform data of the electric signal and the sensor ID from the biological information sensor and transmits the received time-series waveform data of the electric signal and a sensor-related ID related to the received sensor ID without attaching a user ID, which is personal information for identifying the user, to the time-series waveform data of the electric signal and the sensor-related ID. Moreover, a server device receives the time-series waveform data of the electric signal and the sensor-related ID from the relay device and stores the received time-series waveform data of the electric signal and the received sensor-related ID without attaching the user ID, which is the personal information for identifying the user, to the received time-series waveform data of the electric signal and the received sensor-related ID and without processing the time-series waveform data of the electric signal into the biological information that is the medical information as well as the sensitive personal information.

In another exemplary aspect, a relay device is provided that relays information between a biological information sensor and a server device. The relay device is configured to receive time-series waveform data of an electric signal related to biological information of a user and a sensor ID for identifying the biological information sensor from the biological information sensor. In this aspect, the time-series waveform data of the electric signal is medical information unprocessed data that has not been processed into the biological information that is medical information as well as sensitive personal information. The relay device is further configured to transmit, to the server device, the received time-series waveform data of the electric signal and a sensor-related ID related to the received sensor ID without attaching a user ID, which is personal information for identifying the user, to the time-series waveform data of the electric signal and the sensor-related ID.

In yet another exemplary aspect, a biological information management method is provided for managing a measurement result related to biological information of a user and obtained by a biological information sensor. In this aspect, the biological information management method includes a biological information measuring step of measuring time-series waveform data of an electric signal related to the biological information of the user and transmitting the measured time-series waveform data of the electric signal and a sensor ID for identifying the biological information sensor, the time-series waveform data of the electric signal being medical information unprocessed data that has not been processed into the biological information that is medical information as well as sensitive personal information. The method further includes a relaying step of receiving the time-series waveform data of the electric signal and the sensor ID and transmitting the received time-series waveform data of the electric signal and a sensor-related ID related to the received sensor ID without attaching a user ID, which is personal information for identifying the user, to the time-series waveform data of the electric signal and the sensor-related ID. Finally, the method includes a storing step of receiving the time-series waveform data of the electric signal and the sensor-related ID and storing the received time-series waveform data of the electric signal and the received sensor-related ID without attaching the user ID, which is the personal information for identifying the user, to the received time-series waveform data of the electric signal and the received sensor-related ID and without processing the time-series waveform data of the electric signal into the biological information that is the medical information as well as the sensitive personal information.

The exemplary aspects of the present invention improve security in the management of biological information.

### BRIEF DESCRIPTION OF DRAWINGS

FIG. 1 is a diagram illustrating a biological information management system according to the exemplary embodiment.

FIG. 2 is a diagram illustrating a relay device in the biological information management system illustrated in FIG. 1.

FIG. 3 is a diagram illustrating a server device in the biological information management system illustrated in FIG. 1.

FIG. 4 is a diagram illustrating a viewing device in the biological information management system illustrated in FIG. 1.

FIG. 5A is a data transition diagram illustrating examples of a biological information management operation and a biological information viewing operation performed by the biological information management system according to the exemplary embodiment.

FIG. 5B is a data transition diagram illustrating a biological information management operation and a biological information viewing operation according to a first variation that are performed by the biological information management system of the exemplary embodiment.

FIG. 5C is a data transition diagram illustrating a biological information management operation and a biological information viewing operation according to a second variation that are performed by the biological information management system of the exemplary embodiment.

FIG. 5D is a data transition diagram illustrating a biological information management operation and a biological information viewing operation according to a third variation that are performed by the biological information management system of the exemplary embodiment.

FIG. 6A is a diagram showing an example of medical information unprocessed data (e.g., raw data) that has not been processed into biological information, which is medical information as well as sensitive personal information.

FIG. 6B is a diagram showing another example of medical information unprocessed data (raw data) that has not been processed into biological information, which is medical information as well as sensitive personal information.

### DETAILED DESCRIPTION OF EMBODIMENTS

An exemplary embodiment of the present invention is described below with reference to the accompanying drawings. Also, the same reference number is assigned to the same or similar components in the drawings.

### First Exemplary Embodiment

<Biological Information Management System>

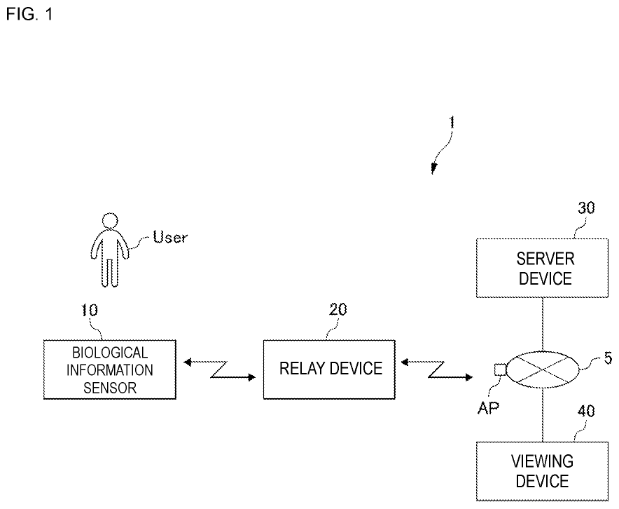

FIG. 1 illustrates a biological information management system according to an exemplary embodiment. As shown, a biological information management system 1 illustrated in FIG. 1 includes a biological information sensor 10, a relay device 20, a server device 30 (also reviewed to as a server), and a viewing device 40 (also reviewed to as a display). In the biological information management system 1, the server device 30 manages a measurement result related to biological information of a user and obtained by the biological information sensor 10. Also, in the biological information management system 1, the biological information of the user can be viewed by using the viewing device 40.

According to an exemplary aspect, the biological information sensor 10 is, for example, a wearable sensor (e.g., a ring sensor) that is configured to be word by the user. In operation, the biological information sensor 10 measures time-series waveform data of an electric signal related to biological information (e.g., vital information or a vital sign) of the user. The biological information sensor 10 transmits the measured time-series waveform data of the electric signal in association with a sensor identifier (ID) that is stored in advance and identifies the biological information sensor. For example, the biological information sensor 10 may include the sensor ID in the time-series waveform data of electric signals as header information in an exemplary aspect. Also, the biological information sensor 10 can be configured to convert the sensor ID into a unique ID through a predetermined calculation and transmit the unique ID. The unique ID obtained by the calculation may also be referred to as a sensor ID.

In exemplary aspects, the biological information sensor 10 may be implemented by, for example, a photoplethysmographic sensor, a heart rate sensor (photoplethysmographic sensor), a carbohydrate sensor (photoplethysmographic sensor), or a core body temperature sensor. The biological information may be, for example, medical information (or sensitive personal information), such as a blood oxygen saturation level SpO2, atrial fibrillation data or arrhythmia data obtained by heart rhythm abnormality detection, a blood sugar level, or a core body temperature. The time-series waveform data of the electric signal is, for example, raw data that has not been processed into the medical information (or sensitive personal information) described above, i.e., medical information (sensitive personal information) unprocessed data on which processing, such as arithmetic processing, has not been performed according to a predetermined algorithm. Details of the biological information sensor 10, the biological information that is medical information (or sensitive personal information), and the time-series waveform data of the electric signal that is medical information (sensitive personal information) unprocessed data are described below.

Furthermore, the relay device 20 can be implemented by, for example, an information processing apparatus such as a smartphone, a tablet, or a PC. The relay device 20 is configured to relay information between the biological information sensor 10 and the server device 30. The relay device 20 receives the time-series waveform data of the electric signal and the sensor ID from the biological information sensor 10. The relay device 20 transmits the received time-series waveform data of the electric signal and a sensor-related ID related to the received sensor ID to the server device 30 via a network 5. The relay device 20 transmits the time-series waveform data of the electric signal and the sensor-related ID as they are without attaching a user identifier (ID), which is considered personal information for identifying the user, to the time-series waveform data and the sensor-related ID. Details of the relay device 20 are discussed below.

According to an exemplary aspect, the server device 30 can be implemented by, for example, an information processing apparatus, such as a PC or a large-scale computer. The server device 30 receives the time-series waveform data of the electric signal and the sensor-related ID from the relay device 20 via the network 5. The server device 30 stores the received time-series waveform data of the electric signal and the received sensor-related ID. Here, the server device 30 stores the time-series waveform data of the electric signal and the sensor-related ID as they are without attaching the user ID, which is personal information for identifying the user, to the time-series waveform data of the electric signal and the sensor-related ID and without processing the time-series waveform data of the electric signal into the biological information, which is medical information as well as sensitive personal information. Details of the server device 30 are described below.

Moreover, the viewing device 40 can be implemented by an information processing apparatus such as a smartphone, a tablet, or a PC. The viewing device 40 can be used by a doctor, for example, providing remote medical care, or the user to view the biological information that is medical information as well as sensitive personal information. The viewing device 40 displays the biological information based on the sensor-related ID. Details of the viewing device 40 are described below.

As a non-limiting example, a near field communication standard, such as Bluetooth® or Wi-Fi®, may be used for communication between the biological information sensor 10 and the relay device 20. Also, as a non-limiting example, a radio communication standard such as a wireless local area network (LAN), Long Term Evolution (LTE), 3G, 4G, or 5G or a wired communication standard, such as a wired LAN, may be used for communication between the relay device 20 and the server device 30 via an access point AP and the network 5. Also, as a non-limiting example, a radio communication standard, such as a wireless local area network (LAN), or a wired communication standard, such as a wired LAN, may be used for communication between the server device 30 and the viewing device 40 via the network 5.

<<Biological Information Sensor>>

The biological information sensor 10 is described in detail below. As described above, the biological information sensor 10 can be configured to measure time-series waveform data of an electric signal related to biological information (e.g., medical information or sensitive personal information) of the user.

For purposes of this disclosure, it should be appreciated that the Act on the Protection of Personal Information provides that medical information described below corresponds to sensitive personal information.

Results of medical examination, etc. performed by, for example, a doctor, and

Guidance, medical treatments, or prescription performed by a doctor, etc. based on the results of medical examination, etc. (Article 2 Paragraph 3 of the Act on the Protection of Personal Information, and Article 2 Item 2 and Item 3 of the Order for Enforcement of the Act on the Protection of Personal Information)

In the present application, biological information indicates information processed into medical information (or sensitive personal information) based on which a doctor can perform medical examination to determine whether the health condition of the user is normal or abnormal, i.e., information processed into medical information (for example, a blood oxygen saturation level SpO2, atrial fibrillation data or arrhythmia data obtained by heart rhythm abnormality detection, a blood sugar level, or a core body temperature) that can be displayed as a result of the medical examination.

On the other hand, time-series waveform data of an electric signal indicates data that has not been processed into medical information (or sensitive personal information), i.e., medical information unprocessed data on which processing, such as arithmetic processing, has not been performed according to a predetermined algorithm for generating medical information. That is, time-series waveform data of an electric signal not only indicates raw data measured by a sensor, but also indicates any type of data that has not been processed into medical information that can be displayed as a result of medical examination. For example, as described later, time-series waveform data of an electric signal may indicate time-series data made up of only peak values, a waveform indicated by time-series data, or data digitized (discretized) by a digital filtering process.

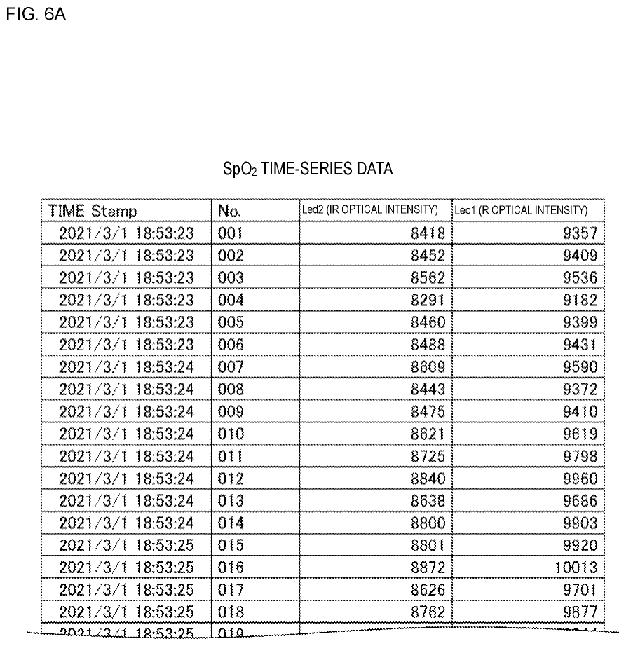

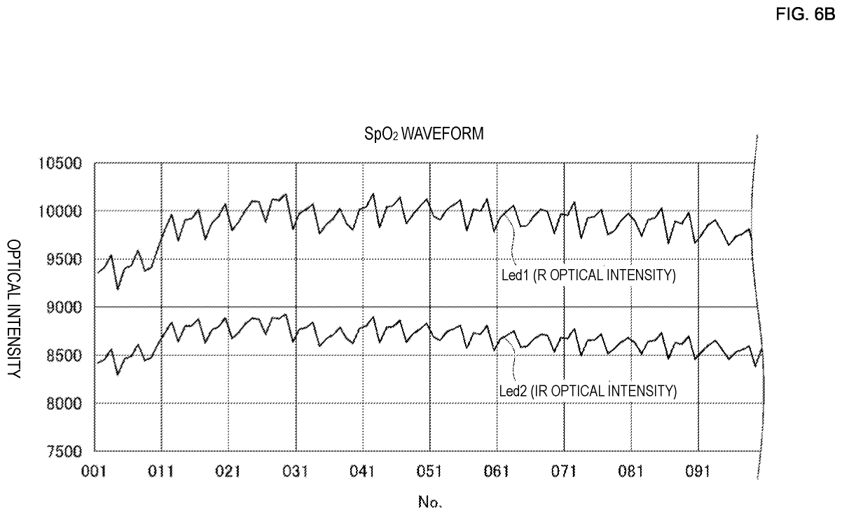

For example, when the biological information sensor 10 is a photoplethysmographic sensor, the photoplethysmographic sensor emits infrared light (IR) and red light (R) toward the user and measures reflected light of the infrared light (IR) and reflected light of the red light (R) that change according to a change in oxygen concentration in the blood or transmitted light of the infrared light (IR) and transmitted light of the red light (R) that change according to a change in oxygen concentration in the blood. As illustrated in FIG. 6A, the photoplethysmographic sensor generates time-series data of electric signals, which correspond to the reflected light or the transmitted light of the infrared light (IR) and the reflected light or the transmitted light of the red light (R), as time-series waveform data of the electric signals. Alternatively, as illustrated in FIG. 6B, the photoplethysmographic sensor generates waveforms indicated by the time-series data of electric signals as time-series waveform data of the electric signals.

By performing arithmetic processing on the time-series waveform data of the electric signals according to a predetermined algorithm, a blood oxygen saturation level SpO2, i.e., biological information, is obtained. This biological information corresponds to medical information, i.e., sensitive personal information, defined in the Act on the Protection of Personal Information. On the other hand, the time-series waveform data of the electric signals described above is raw data (medical information unprocessed data) that has not been processed into biological information or medical information and does not correspond to medical information or sensitive personal information defined in the Act on the Protection of Personal Information.

The photoplethysmographic sensor may also be used as a heart rate sensor. For example, when the biological information sensor 10 is a heart rate sensor, the heart rate sensor measures peaks of reflected light of infrared light (IR) and reflected light of red light (R) that vary depending on changes in the blood flow rate caused by the heartbeat. The heart rate sensor generates time-series waveform data, i.e., pulse wave data or heart rate data, of the peaks of electric signals corresponding to the peaks of the reflected light of the infrared light (IR) and the reflected light of the red light (R).

By performing arithmetic processing on the time-series waveform data of these electric signals according to a predetermined algorithm, atrial fibrillation data or arrhythmia data, i.e., biological information, indicating results of heart rhythm abnormality detection is obtained. This biological information corresponds to medical information, i.e., sensitive personal information, defined in the Act on the Protection of Personal Information as described above. On the other hand, the time-series waveform data of the electric signals described above is raw data (e.g., medical information unprocessed data) that has not been processed into biological information or medical information and does not correspond to medical information or sensitive personal information defined in the Act on the Protection of Personal Information.

The photoplethysmographic sensor is also used as a carbohydrate sensor. For example, when the biological information sensor 10 is a carbohydrate sensor, the carbohydrate sensor measures the pulse waveform of reflected light or transmitted light that changes depending on the carbohydrate concentration in the blood. The carbohydrate sensor generates a waveform, i.e., time-series waveform data, of an electric signal corresponding to the pulse waveform of reflected light or transmitted light.

In operation, arithmetic processing is performed on the waveform or the time-series waveform data of the electric signal according to a predetermined algorithm to obtain a blood sugar level, i.e., biological information. This biological information corresponds to medical information, i.e., sensitive personal information, defined in the Act on the Protection of Personal Information. On the other hand, the time-series waveform data of the electric signal described above is raw data (e.g., medical information unprocessed data) that has not been processed into biological information or medical information and does not correspond to medical information or sensitive personal information defined in the Act on the Protection of Personal Information.

Also, for example, when the biological information sensor 10 is a core body temperature sensor, the core body temperature sensor measures time-series waveform data of electric signals (electric signals corresponding to temperatures) received from multiple temperature sensors (e.g., thermistors) arranged at various intervals on the body surface of the user.

Arithmetic processing is performed on the time-series waveform data of the electric signals according to a predetermined algorithm to obtain a core body temperature, i.e., biological information. This biological information corresponds to medical information, i.e., sensitive personal information, defined in the Act on the Protection of Personal Information. On the other hand, the time-series waveform data of the electric signals described above is raw data (e.g., medical information unprocessed data) that has not been processed into biological information or medical information and does not correspond to medical information or sensitive personal information defined in the Act on the Protection of Personal Information.

<<Relay Device>>

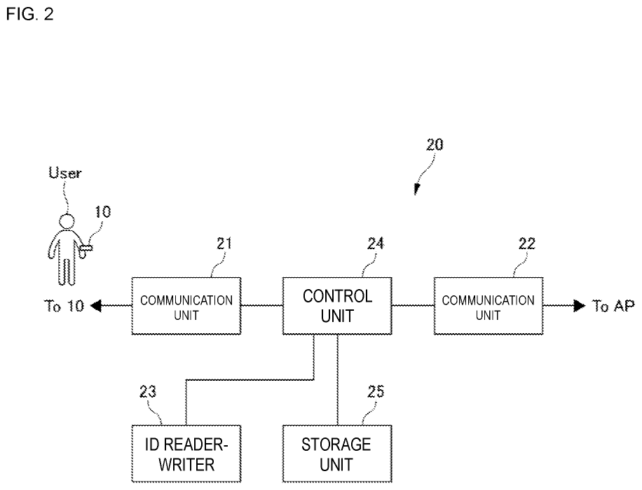

The relay device 20 is described in detail below. FIG. 2 illustrates the relay device in the biological information management system illustrated in FIG. 1. The relay device 20 illustrated in FIG. 2 includes communication units 21 and 22, an ID reader-writer 23, a control unit 24, and a storage unit 25.

According to the exemplary aspect, the communication unit 21 is configured to wirelessly communicate with the biological information sensor 10. The communication unit 21 is an interface that is compliant with a near field communication standard, such as Bluetooth® or Wi-Fi®. It should be appreciated that the communication unit 21 may also be compliant with any other communication standard.

The communication unit 22 communicates with the server device 30 via, for example, the access point AP and the network 5. The communication unit 22 is an interface that performs wireless communication according to a communication standard, such as a wireless local area network (LAN), Long Term Evolution (LTE), 3G, 4G, or 5G.

The ID reader-writer 23 is configured to read an identifier (ID) recorded in a recording medium and writes an ID to the recording medium. The ID reader-writer 23 is implemented by, for example, an ID card reader-writer or a radio frequency identifier (RFID) reader-writer, and the recording medium is, for example, an ID card or an RFID tag.

The control unit 24 is configured to control the entire operation of the relay device 20. For example, based on a user ID and a sensor-related ID read by the ID reader-writer 23 or based on a sensor-related ID stored in advance in the storage unit 25, the control unit 24 receives time-series waveform data of an electric signal and a sensor ID from the biological information sensor 10 corresponding to the sensor-related ID via the communication unit 21. The control unit 24 transmits the received time-series waveform data of the electric signal and the sensor-related ID related to the received sensor ID to the server device 30 via the communication unit 22. Here, the control unit 24 transmits the time-series waveform data of the electric signal and the sensor-related ID as they are without attaching the user ID, which is personal information for identifying the user, to the time-series waveform data of the electric signal and the sensor-related ID.

For purposes of this disclosure, the sensor-related ID can be the sensor ID itself or can be a unique ID obtained by performing predetermined basic arithmetic operations between the sensor ID and the user ID. Moreover, the control unit 24 can be configured to cause the ID reader-writer 23 to write the generated sensor-related ID to the recording medium.

According to the exemplary aspect, the storage unit 25 is configured to store programs and/or applications to be executed by the control unit 24 or data. In particular, the storage unit 25 may store, in advance, the sensor-related ID of the biological information sensor 10 available to the user or may store a generated sensor-related ID. The storage unit 25 may be implemented by a storage medium such as a read-only memory (ROM), a hard disk drive (HDD), a solid-state drive (SSD), or a removable memory card.

The control unit 24 described above is implemented by, for example, an arithmetic processor such as a central processing unit (CPU), a digital signal processor (DSP), or a field-programmable gate array (FPGA). For example, various functions of the control unit 24 are implemented by executing the programs and/or applications stored in the storage unit 25. The programs and/or applications may be provided via a network or via a computer-readable storage medium such as a compact disc read-only memory (CD-ROM) or a digital versatile disk (DVD) storing the programs. The storage medium is, for example, a non-transitory tangible medium.

<<Server Device>>

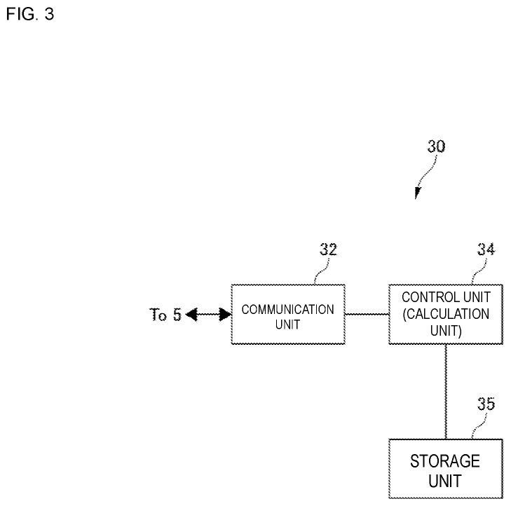

The server device 30 is described in detail below. FIG. 3 illustrates the server device in the biological information management system illustrated in FIG. 1. The server device 30 illustrated in FIG. 3 includes a communication unit 32, a control unit 34, and a storage unit 35.

The communication unit 32 is configured to communicate with the relay device 20 and the viewing device 40 via, for example, the network 5. The communication unit 32 is an interface that performs wireless communication according to a radio communication standard such as a wireless local area network (LAN) or a communication standard such as a wired LAN.

The control unit 34 controls the entire operation of the server device 30. The control unit 34 receives time-series waveform data of an electric signal and a sensor-related ID from the relay device 20 via the communication unit 32. Moreover, the control unit 34 stores the received time-series waveform data of the electric signal and the received sensor-related ID in the storage unit 35. According to the exemplary aspect, the control unit 34 stores the time-series waveform data of the electric signal and the sensor-related ID in the storage unit 35 as they are without attaching a user ID, which is personal information for identifying the user, to the time-series waveform data of the electric signal and the sensor-related ID and without processing the time-series waveform data of the electric signal into biological information that is medical information as well as sensitive personal information.

In response to a request received from the viewing device 40 via the communication unit 32, the control unit 34 transmits the time-series waveform data of the electric signal that is stored in the storage unit 35 and corresponds to a sensor-related ID in the request to the viewing device 40 via the communication unit 32 without processing the time-series waveform data of the electric signal into biological information that is medical information as well as sensitive personal information.

Alternatively, the control unit 34 can be configured to function as a calculation unit and process the time-series waveform data of the electric signal into biological information that is medical information as well as sensitive personal information according to a predetermined algorithm stored in advance in the storage unit 35. In this case, in response to a request received from the viewing device 40, the control unit 34 may process the time-series waveform data of the electric signal stored in the storage unit 35 and corresponding to the user-related ID in the request to obtain biological information and transmit the obtained biological information. In this case, the control unit 34 does not store the obtained biological information, which is medical information as well as sensitive personal information, in the storage unit 35.

The storage unit 35 is configured to store programs (e.g., software applications) to be executed by the control unit 34 or data. The storage unit 35 also stores the received time-series waveform data of the electric signal and the received sensor-related ID. The storage unit 35 may also store the predetermined algorithm for processing the time-series waveform data of the electric signal into biological information or medical information. According to an exemplary aspect, the storage destination address of the time-series waveform data of the electric signal is preferably different from the storage destination address of the predetermined algorithm. The storage unit 35 is implemented by a storage medium such as a read only memory (ROM), a hard disk drive (HDD), a solid-state drive (SSD), or a removable memory card.

According to an exemplary aspect, the control unit 34, as described above, can be implemented by, for example, an arithmetic processor such as a central processing unit (CPU), a digital signal processor (DSP), or a field-programmable gate array (FPGA). For example, various functions of the control unit 34 are implemented by executing the programs and/or software applications stored in the storage unit 35. The programs and/or applications may be provided via a network or via a computer-readable storage medium such as a compact disc read-only memory (CD-ROM) or a digital versatile disk (DVD) storing the programs. The storage medium is, for example, a non-transitory tangible medium.

<<Viewing Device>>

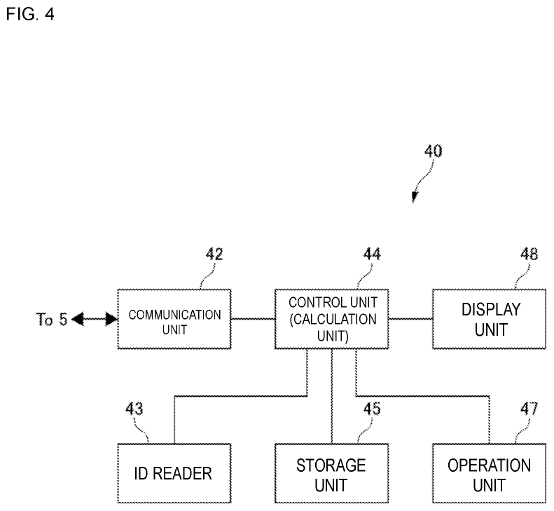

The viewing device 40 is described in detail below. FIG. 4 illustrates the viewing device in the biological information management system illustrated in FIG. 1. The viewing device 40 illustrated in FIG. 4 includes a communication unit 42, an ID reader 43, a control unit 44, a storage unit 45, an operation unit 47, and a display unit 48.

The communication unit 42 communicates with the server device 30 via, for example, the network 5. The communication unit 42 is an interface that performs wireless communication according to a radio communication standard such as a wireless local area network (LAN) or a communication standard such as a wired LAN.

The ID reader 43 reads an identifier (ID) recorded in a recording medium. The ID reader 43 is implemented by, for example, an ID card reader or a radio frequency identifier (RFID) reader, and the recording medium is, for example, an ID card or an RFID tag.

The operation unit 47 is used by the user to perform operations. The operation unit 47 is implemented by, for example, a keyboard or a mouse having physical operation buttons or a touch panel having virtual operation buttons.

The display unit 48 displays biological information and can be implemented by, for example, a liquid crystal display or an organic EL display.

Moreover, the control unit 44 is configured to control the entire operation of the viewing device 40. The control unit 44 enables viewing of biological information based on a user ID or an administrator ID different from the user ID input via the operation unit 47. Based on the user-related ID input by the user via the operation unit 47, the control unit 44 transmits a request for the corresponding biological information to the server device 30 via the communication unit 42.

Alternatively, the control unit 44 can enable viewing of biological information based on a user ID or an administrator ID different from the user ID that is read from a recording medium by the ID reader 43. Also, the control unit 44 may be configured to transmit, based on a sensor-related ID read from a recording medium by the ID reader 43, a request for the corresponding biological information to the server device 30 via the communication unit 42.

In operation, the control unit 44 receives the time-series waveform data of the electric signal and the user-related ID from the server device 30 via the communication unit 42. The control unit 44 can be configured to function as a calculation unit and processes the time-series waveform data of the electric signal into biological information, which is medical information as well as sensitive personal information, according to a predetermined algorithm stored in advance in the storage unit 45. Alternatively, the control unit 44 may process the time-series waveform data of the electric signal to obtain biological information, which is medical information as well as sensitive personal information, according to a predetermined algorithm read by the ID reader 43. The control unit 44 displays the obtained biological information on the display unit 48.

Alternatively, when biological information is obtained at the server device 30, the control unit 44 may receive the biological information from the server device 30 and display the received biological information on the display unit 48.

The storage unit 45 stores programs and/or applications to be executed by the control unit 44 or data. The storage unit 45 may also store a predetermined algorithm for processing time-series waveform data of an electric signal into biological information (e.g., medical information or sensitive personal information). The storage unit 45 may also store the time-series waveform data of the electric signal and the user-related ID that have been received, the biological information and the user-related ID that have been received, or the obtained biological information and the user-related ID. The storage unit 45 is implemented by a storage medium such as a read-only memory (ROM), a hard disk drive (HDD), a solid-state drive (SSD), or a removable memory card.

As described above, the control unit 44 can be implemented by, for example, an arithmetic processor such as a central processing unit (CPU), a digital signal processor (DSP), or a field-programmable gate array (FPGA). For example, various functions of the control unit 44 are implemented by executing the programs and/or applications stored in the storage unit 45. The programs and/or applications may be provided via a network or via a computer-readable storage medium such as a compact disc read-only memory (CD-ROM) or a digital versatile disk (DVD) storing the programs. The storage medium is, for example, a non-transitory tangible medium.

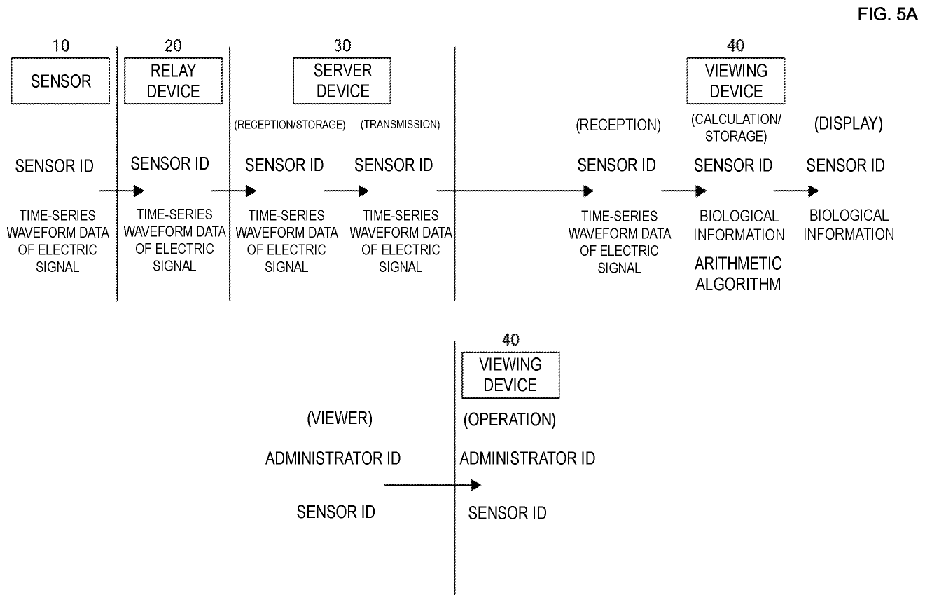

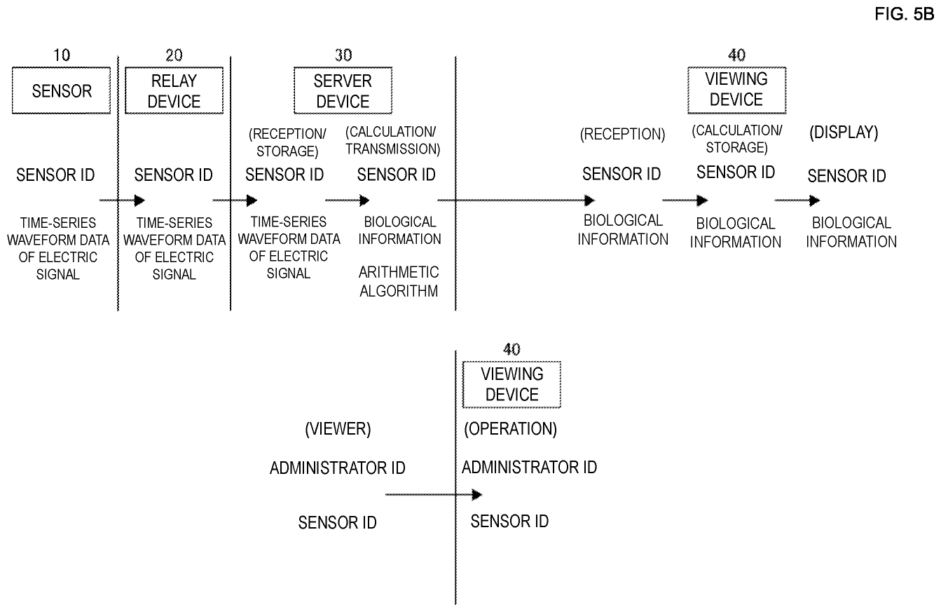

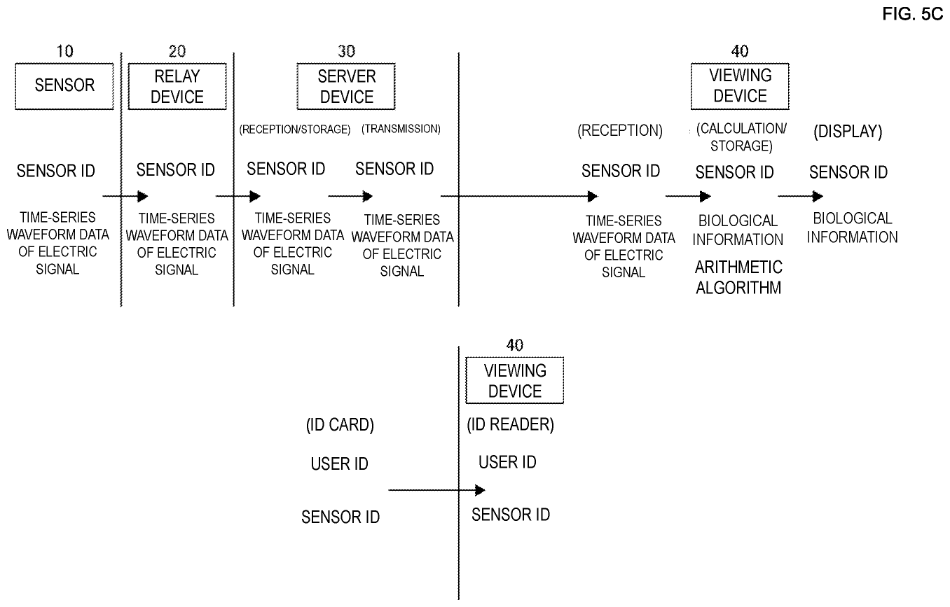

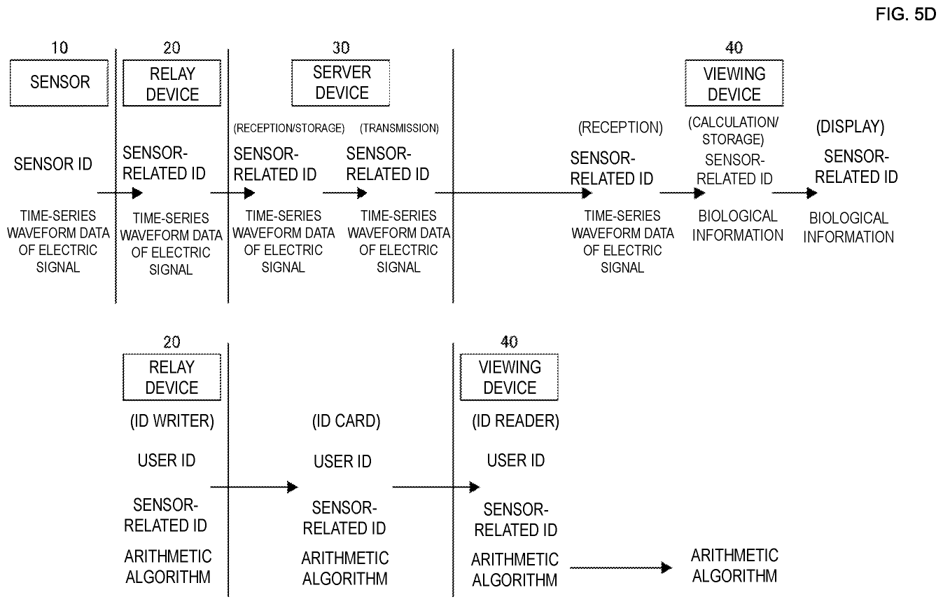

Next, with reference to FIG. 5A, a biological information management operation and a biological information viewing operation performed by the biological information management system 1 are described. FIG. 5A is a data transition diagram illustrating examples of a biological information management operation and a biological information viewing operation performed by the biological information management system according to the exemplary embodiment.

First, the biological information management operation is described. The biological information sensor 10 is configured to measure time-series waveform data of an electric signal related to biological information (e.g., medical information or sensitive personal information) of the user. As described above, the time-series waveform data of the electric signal is medical information unprocessed data (or raw data) that has not been processed into biological information that is medical information (or sensitive personal information). The biological information sensor 10 transmits the measured time-series waveform data of the electric signal and a prestored sensor ID (e.g., a biological information measuring step).

The relay device 20 receives the time-series waveform data of the electric signal and the sensor ID from the biological information sensor 10 and transmits the time-series waveform data of the electric signal and the sensor ID that have been received. Here, the relay device 20 transmits the time-series waveform data of the electric signal and the sensor ID as they are without attaching a user ID, which is personal information for identifying the user, to the time-series waveform data of the electric signal and the sensor ID (e.g., a relaying step).

The server device 30 receives the time-series waveform data of the electric signal and the sensor ID from the relay device 20 and stores the time-series waveform data of the electric signal and the sensor ID that have been received. Here, the server device 30 stores the time-series waveform data of the electric signal and the sensor ID as they are without attaching the user ID, which is personal information for identifying the user, to the time-series waveform data of the electric signal and the sensor ID and without processing the time-series waveform data of the electric signal into biological information that is medical information (or sensitive personal information) (e.g., a storing step).

Next, the biological information viewing operation is described. A viewer, such as a doctor or a user, operates the operation unit 47 of the viewing device 40 to enter an administrator ID and a sensor ID. The viewing device 40 then enables viewing of the biological information based on the entered administrator ID. Also, based on the entered sensor ID, the viewing device 40 transmits a request for the corresponding biological information to the server device 30.

In response to the request from the viewing device 40, the server device 30 transmits the time-series waveform data of the electric signal that is stored in the server device 30 and corresponds to the sensor ID in the request to the viewing device 40 without processing the time-series waveform data of the electric signal into biological information that is medical information (or sensitive personal information).

Then, the viewing device 40 receives the time-series waveform data of the electric signal and the sensor ID from the server device 30 and performs arithmetic processing on the time-series waveform data of the electric signal according to a predetermined algorithm prestored in the viewing device 40 to obtain biological information that is medical information (or sensitive personal information). The viewing device 40 displays the obtained biological information (e.g., a viewing step). The viewing device 40 may also store the obtained biological information.

As described above, in the biological information management system 1 of the exemplary embodiment, the relay device transmits time-series waveform data of an electric signal that is related to biological information and measured by the biological information sensor, i.e., medical information unprocessed data (or raw data) that has not been processed into biological information that is medical information as well as sensitive personal information. The server device stores the time-series waveform data of the electric signal received from the relay device without processing the time-series waveform data of the electric signal into biological information that is medical information as well as sensitive personal information. With this configuration, medical information, which is sensitive personal information, is not generated by the relay device and the server device. Accordingly, even when the relay device and the server device are accessed by a malicious third party, this configuration prevents leaking, unauthorized viewing (or peeping), and tampering of medical information, which is sensitive personal information, and thereby improves security.

According to an exemplary aspect, even medical information unprocessed data (or raw data), which is not medical information nor sensitive personal information, becomes sensitive personal information when the medical information unprocessed data is stored together with a user ID that is personal information for identifying the user. For example, when the relay device is a mobile device, such as a smartphone, a tablet, or a PC, personal information (for example, information, such as a name, a birth date, and/or a gender, with which the user can be identified) may be recorded in the mobile device or registered in an application that operates in conjunction with the relay device. In such a case, i.e., when medical information unprocessed data (or raw data), which is not medical information nor sensitive personal information, exists together with personal information, the medical information unprocessed data still corresponds to sensitive personal information.

For this reason, in the biological information management system 1 of the exemplary embodiment, the relay device does not transmit a user ID, which is personal information for identifying the user, and instead transmits a sensor ID for identifying the sensor. Also, the server device does not store the user ID, which is personal information for identifying the user, and instead stores the sensor ID for identifying the sensor. With this configuration, the medical information unprocessed data (or raw data) and the user ID do not exist together in the relay device and the server device. Accordingly, even when the relay device or the server device is accessed by a malicious third party, because the medical information unprocessed data (or raw data) alone does not correspond to sensitive personal information, the above configuration prevents leaking, unauthorized viewing (or peeping), and tampering of sensitive personal information and thereby improves security.

In the biological information management system 1 of the exemplary embodiment, the viewing device indirectly identifies biological information of a desired user by using the sensor ID instead of the user ID, which is personal information for identifying the user, and displays and stores the identified biological information. Even when the viewing device is accessed by a malicious third party, this configuration prevents the malicious third party from identifying the user to which the biological information, which is medical information as well as sensitive personal information, belongs.

Furthermore, according to the biological information management system 1 of the exemplary embodiment, even when the viewing device is accessed by a malicious third party by logging into the viewing device using an administrator ID different from the user ID, which is personal information for identifying the user, the malicious third party is prevented from identifying the user to which the biological information, which is medical information as well as sensitive personal information, belongs.

### First Variation of the Exemplary Embodiment

In the embodiment described above, the viewing device 40 stores a predetermined algorithm in advance and processes time-series waveform data of an electric signal into biological information that is medical information (or sensitive personal information). However, the exemplary embodiment is not limited to this example. In another aspect, the server device 30 can be configured to store a predetermined algorithm in advance and process the time-series waveform data of the electric signal into biological information, which is medical information (or sensitive personal information), in response to a request from the viewing device 40.

FIG. 5B is a data transition diagram illustrating a biological information management operation and a biological information viewing operation according to a first variation that are performed by the biological information management system of the exemplary embodiment. It is noted that the biological information management operation according to the first variation illustrated in FIG. 5B is the same as the above-described biological information management operation illustrated in FIG. 5A.

The biological information viewing operation according to the first variation illustrated in FIG. 5B is described. As described in the above embodiment, a viewer, such as a doctor or a user, operates the operation unit 47 of the viewing device 40 to enter an administrator ID and a sensor ID. The viewing device 40 then enables viewing of biological information based on the entered administrator ID. Also, based on the entered sensor ID, the viewing device 40 transmits a request for the corresponding biological information to the server device 30.

In response to the request from the viewing device 40, the server device 30 selects time-series waveform data of an electric signal that is stored in the server device 30 and corresponds to the user ID in the request. The server device 30 processes the time-series waveform data of the electric signal to obtain biological information, which is medical information (or sensitive personal information), according to the predetermined algorithm stored in advance. The server device 30 transmits the obtained biological information to the viewing device 40. In this case, the server device 30 does not store the obtained biological information that is medical information (or sensitive personal information).

The viewing device 40 displays the biological information and the sensor ID received from the server device 30 (e.g., a viewing step). The viewing device 40 may also store the received biological information.

The first variation also has advantageous effects similar to those of the biological information management system 1 of the exemplary embodiment described above.

### Second Variation of the Exemplary Embodiment

In the embodiment described above, when viewing biological information, a viewer, such as a doctor or a user, manually enters an administrator ID and a sensor ID by operating the operation unit 47 of the viewing device 40. However, the exemplary embodiment is not limited to this example. In another aspect, a viewer, such as a doctor or a user, may automatically enter a user ID and a sensor ID using a recording medium such as an ID card or an RFID tag.

FIG. 5C is a data transition diagram illustrating a biological information management operation and a biological information viewing operation according to a second variation that are performed by the biological information management system of the exemplary embodiment. The biological information management operation according to the second variation illustrated in FIG. 5C is the same as the above-described biological information management operation illustrated in FIG. 5A.

The biological information viewing operation according to the second variation illustrated in FIG. 5C is described. For example, a viewer, such as a doctor or a user, sets an ID card in the ID reader 43 of the viewing device 40. In response, the viewing device 40 automatically recognizes a user ID and a sensor ID recorded in the ID card. Then, the viewing device 40 enables viewing of biological information based on the recognized user ID. Also, based on the recognized sensor ID, the viewing device 40 transmits a request for the corresponding biological information to the server device 30.

Similarly to the exemplary embodiment described above, in response to the request from the viewing device 40, the server device 30 transmits the time-series waveform data of the electric signal that is stored in the server device 30 and corresponds to the user ID in the request to the viewing device 40 without processing the time-series waveform data of the electric signal into biological information that is medical information (or sensitive personal information).

Similarly to the exemplary embodiment described above, the viewing device 40 receives the time-series waveform data of the electric signal and the sensor ID from the server device 30 and performs arithmetic processing on the time-series waveform data of the electric signal according to a predetermined algorithm stored in advance to obtain biological information that is medical information (or sensitive personal information). The viewing device 40 displays the obtained biological information (e.g., a viewing step). The viewing device 40 may also store the obtained biological information.

The second variation also has advantageous effects similar to those of the biological information management system 1 of the exemplary embodiment described above.

Furthermore, the second variation, for example, eliminates the need to rely on the user's memory of the user ID in viewing the biological information and thereby improves convenience. Also, performing a reference check by using, for example, an ID card prevents unauthorized use by impersonation using the user ID.

### Third Variation of the Exemplary Embodiment

In the embodiment and the variations described above, the sensor ID itself is used to identify biological information. However, it should be appreciated that this disclosure is not limited to the embodiment and the variations described above, and a sensor-related ID related to the sensor ID may also be used to identify biological information. Also, in the second variation described above, a recording medium such as an ID card may record a predetermined algorithm for processing time-series waveform data of an electric signal into biological information that is medical information (or sensitive personal information).

FIG. 5D is a data transition diagram illustrating a biological information management operation and a biological information viewing operation according to a third variation that are performed by the biological information management system of the exemplary embodiment.

First, the biological information management operation is described. Similarly to the exemplary embodiment described above, the biological information sensor 10 measures time-series waveform data of an electric signal related to biological information (e.g., medical information or sensitive personal information) of the user and transmits the measured time-series waveform data of the electric signal and a prestored sensor ID (e.g., a biological information measuring step).

The relay device 20 receives the time-series waveform data of the electric signal and the sensor ID from the biological information sensor 10. The relay device 20 transmits the received time-series waveform data of the electric signal and a sensor-related ID related to the received sensor ID. Here, the relay device 20 transmits the time-series waveform data of the electric signal and the sensor-related ID without attaching a user ID, which is personal information for identifying the user, to the time-series waveform data of the electric signal and the sensor-related ID (e.g., a relaying step).

Here, the sensor-related ID is a unique ID obtained by performing predetermined basic arithmetic operations between the sensor ID and the user ID. The relay device 20 writes the generated sensor-related ID to the ID card using the ID reader-writer 23.

The server device 30 receives the time-series waveform data of the electric signal and the sensor-related ID from the relay device 20 and stores the time-series waveform data of the electric signal and the sensor-related ID that have been received. Here, the server device 30 stores the time-series waveform data of the electric signal and the sensor-related ID as they are without attaching the user ID, which is personal information for identifying the user, to the time-series waveform data of the electric signal and the sensor-related ID and without processing the time-series waveform data of the electric signal into biological information that is medical information (or sensitive personal information) (e.g., a storing step).

Next, the biological information viewing operation is described. For example, a viewer, such as a doctor or a user, sets an ID card in the ID reader 43 of the viewing device 40. In response, the viewing device 40 automatically recognizes a user ID, a sensor-related ID, and a predetermined algorithm for processing time-series waveform data of an electric signal into biological information or medical information (or sensitive personal information) that are recorded in the ID card. Then, the viewing device 40 enables viewing of biological information based on the recognized user ID. Also, based on the recognized sensor-related ID, the viewing device 40 transmits a request for the corresponding biological information to the server device 30.

In response to the request from the viewing device 40, the server device 30 transmits time-series waveform data of an electric signal that is stored in the server device 30 and corresponds to the user-related ID in the request to the viewing device 40 without processing the time-series waveform data of the electric signal into biological information that is medical information (or sensitive personal information).

The viewing device 40 receives the time-series waveform data of the electric signal and the sensor-related ID from the server device 30 and performs arithmetic processing on the time-series waveform data of the electric signal according to the predetermined algorithm read from the ID card to obtain biological information that is medical information (or sensitive personal information). The viewing device 40 displays the obtained biological information (e.g., a viewing step). The viewing device 40 may also store the obtained biological information.

It should be appreciated that the third variation also has advantageous effects similar to those of the second variation described above.

According to the third variation, instead of the sensor ID itself, the sensor-related ID, which is a unique ID obtained by performing predetermined basic arithmetic operations between the sensor ID and the user ID, is used to identify biological information. With this configuration, both of the user ID, which is personal information for identifying the user, and the sensor ID do not exist in the relay device 20 and the server device 30. Thus, this configuration improves confidentiality.

Also, according to the third variation, the relay device 20 can additionally be configured to write the generated sensor-related ID to the ID card so that the sensor-related ID can be used when viewing biological information. Accordingly, the third variation further improves convenience of the exemplary system and methodology.

Also, according to the third variation, the ID card can be configured to record a predetermined algorithm for processing time-series waveform data of an electric signal into biological information, which is medical information (or sensitive personal information), and the viewing device 40 reads the predetermined algorithm recorded in the ID card when viewing the biological information. This configuration eliminates the need to store the predetermined algorithm in advance in the viewing device 40 and thereby prevents the reverse analysis of algorithm calculations.

In general, it is noted that exemplary embodiments of the present invention are described above. However, the present invention is not limited to the above-described embodiments, and the embodiments may be modified, transformed, and combined in various manners. In the above embodiments and variations, four examples are described with reference to FIGS. 5A through 5D. However, the present invention is not limited to these examples, and all or some of the four examples illustrated in FIGS. 5A through 5D may be combined.

### REFERENCE SIGNS LIST

1 biological information management system
5 network
10 biological information sensor
20 relay device
21, 22 communication unit
23 ID reader-writer
24 control unit
25 storage unit
30 server device
32 communication unit
34 control unit
35 storage unit
40 viewing device
42 communication unit
43 ID reader
44 control unit
45 storage unit
47 operation unit
48 display unit
AP access point

1. A biological information management system comprising:
a biological information sensor configured to measure time-series waveform data of an electric signal related to biological information of a user and to transmit the measured time-series waveform data of the electric signal and a sensor ID that identifies the biological information sensor;
a relay device configured to receive the time-series waveform data of the electric signal and the sensor ID from the biological information sensor and to transmit the received time-series waveform data of the electric signal and a sensor-related ID related to the received sensor ID without a user ID that identifies the user; and
a server configured to receive the time-series waveform data of the electric signal and the sensor-related ID from the relay device and to store the received time-series waveform data of the electric signal and the received sensor-related ID without attaching the user ID to the received time-series waveform data of the electric signal and the received sensor-related ID.

2. The biological information management system according to claim 1, further comprising:
a viewing device configured to display, based on the sensor-related ID, the biological information including medical information and sensitive personal information,
wherein, in response to a request from the viewing device, the server is configured to transmit the time-series waveform data of the electric signal corresponding to the sensor-related ID in the request without processing the time-series waveform data of the electric signal into the biological information, and
wherein the viewing device is configured to process the time-series waveform data of the electric signal received from the server into the biological information, which is displayed thereon.

3. The biological information management system according to claim 1, further comprising:
a viewing device configured to display, based on the sensor-related ID, the biological information that includes medical information and sensitive personal information,
wherein, in response to a request from the viewing device, the server is configured to process the time-series waveform data of the electric signal corresponding to the sensor-related ID in the request to obtain the biological information and to transmit the obtained biological information without storing the obtained biological information, and
wherein the viewing device is configured to display the biological information received from the server.

4. The biological information management system according to claim 2, wherein the viewing device is further configured to display the biological information based on an administrator ID that is different from the user ID.

5. The biological information management system according to claim 2, wherein the viewing device includes an ID reader that reads the sensor-related ID from a recording medium recording the sensor-related ID.

6. The biological information management system according to claim 2,
wherein the viewing device includes an ID reader configured to read the sensor-related ID and a predetermined algorithm from a recording medium that records the sensor-related ID and the predetermined algorithm for processing the time-series waveform data of the electric signal into the biological information that includes the medical information and the sensitive personal information, and
wherein the viewing device is configured to process the time-series waveform data of the electric signal received from the server into the biological information according to the predetermined algorithm.

7. The biological information management system according to claim 6, wherein:
the recording medium is further configured to record the user ID or an administrator ID that is different from the user ID,
the ID reader is further configured to read the user ID or the administrator ID from the recording medium, and
the viewing device is further configured to display the biological information based on the user ID or the administrator ID.

8. The biological information management system according to claim 1, wherein the sensor-related ID is the sensor ID.

9. The biological information management system according to claim 1, wherein the sensor-related ID is a unique ID obtained by performing predetermined arithmetic operations between the sensor ID and the user ID.

10. The biological information management system according to claim 6, wherein:
the sensor-related ID is a unique ID that is obtained by performing predetermined arithmetic operations between the sensor ID and the user ID, and
the relay device includes an ID writer that is configured to write the sensor-related ID to the recording medium.

11. A relay device that relays information between a biological information sensor and a server, the relay device comprising:
a memory; and
a processor that, when executing instructions stored on the memory, configures the relay device to:
receive time-series waveform data of an electric signal related to biological information of a user and a sensor ID for identifying the biological information sensor from the biological information sensor, and
transmit, to the server, the received time-series waveform data of the electric signal and a sensor-related ID related to the received sensor ID without attaching a user ID that identifies the user.

12. The relay device according to claim 11, wherein the sensor-related ID is the sensor ID.

13. The relay device according to claim 11, wherein the sensor-related ID is a unique ID that is obtained by performing predetermined arithmetic operations between the sensor ID and the user ID.

14. The relay device according to claim 13, further comprising an ID writer configured to generate the sensor-related ID and to write the generated sensor-related ID to a recording medium.

15. A biological information management method comprising:
measuring, by a biological information sensor, time-series waveform data of an electric signal related to biological information of a user;
transmitting, by the biological information sensor, the measured time-series waveform data of the electric signal and a sensor ID for identifying the biological information sensor;
receiving, by a relay device, the time-series waveform data of the electric signal and the sensor ID;
transmitting, by the relay device, the received time-series waveform data of the electric signal and a sensor-related ID related to the received sensor ID without attaching a user ID that identifies the user;
receiving, by a server, the time-series waveform data of the electric signal and the sensor-related ID; and
storing, by the server, the received time-series waveform data of the electric signal and the received sensor-related ID without attaching the user ID to the received time-series waveform data of the electric signal and the received sensor-related ID.

16. The biological information management method according to claim 15, further comprising:
displaying, by a viewing device and based on the sensor-related ID, the biological information including medical information and sensitive personal information;
in response to a request from the viewing device, transmitting, by the server, the time-series waveform data of the electric signal corresponding to the sensor-related ID in the request without processing the time-series waveform data of the electric signal into the biological information; and
processing, by the viewing device, the time-series waveform data of the electric signal received from the server into the biological information that is displayed thereon.

17. The biological information management method according to claim 15, further comprising:
displaying, by a viewing device and based on the sensor-related ID, the biological information that includes medical information and sensitive personal information;
in response to a request from the viewing device, processing, by the server, the time-series waveform data of the electric signal corresponding to the sensor-related ID in the request to obtain the biological information;
transmitting, by the server, the obtained biological information without storing the obtained biological information; and
displaying, by the viewing device, the biological information received from the server.

18. The biological information management method according to claim 16, further comprising displaying, by the viewing device, the biological information based on an administrator ID that is different from the user ID.

19. The biological information management method according to claim 16, further comprising reading, by a reader of the viewing device, the sensor-related ID from a recording medium recording the sensor-related ID.

20. The biological information management method according to claim 16, further comprising:
reading, by an ID reader of the viewing device, the sensor-related ID and a predetermined algorithm from a recording medium that records the sensor-related ID and the predetermined algorithm for processing the time-series waveform data of the electric signal into the biological information that includes the medical information and the sensitive personal information; and
processing, by the viewing device, the time-series waveform data of the electric signal received from the server into the biological information according to the predetermined algorithm.
